(19) United States
(12) Patent Application Publication
(10) Pub. No.: US 2019/0135669 A1
(43) Pub. Date: May 9, 2019

(54) ENHANCED BIOLOGICAL TREATMENT OF WASTEWATER

(71) Applicant: Steven L. Cort, Cary, NC (US)

(72) Inventor: Steven L. Cort, Cary, NC (US)

(21) Appl. No.: 15/732,401

(22) Filed: Nov. 7, 2017

(51) Int. Cl. C02F 3/34; C12N 1/20

(52) U.S. Cl. CPC C02F 3/348; C02F 3/341; C12N 1/20; C02F 2103/007; C02F 2303/04; C02F 2209/40; C02F 2305/06

(57) ABSTRACT

The present invention relates to a new and novel process that combines several water treatment processes to biologically treat wastewater. The first treatment process is bioaugmentation using a select mixture of microorganisms grown from wastes collected from a Waste Water Treatment Plant (WWTP) to treat wastewater as it flows through its conveyance system. The second treatment process is employment of a flow control device that increases the holding capacity of the conveyance system to increase time for biological treatment and allow the contained water to be discharged at a controlled reduced and leveled rate. The third treatment process is a high-rate clarification system preferably using flocculating polymers to remove fine suspended solids from the wastewater that can be further treated in a series of containment structures positioned in a stream or riverbed to increase the treatment residence time.

### FIELD OF THE INVENTION

The present invention relates to the process of processing solid wastes from a Waste Water Treatment Plant (WWTP) that receives sanitary waste from human activity to grow select microorganisms that are used to enhance the operation of the WWTP and to biologically treat wastewater problems typically associated but not limited to wet weather events.

### BACKGROUND OF THE INVENTION

There are many problems associated with wet weather events such as hurricanes. A primary problem is treatment of polluted water, which may be presented in the form of Combined Sewer Overflows (CSO), Separate Sewer Overflows (SSO), and storm water.

Treatment of CSO and other water pollution associated with wet weather events is primarily a biological treatment process that needs certain elements to perform effectively.

A major problem with wet weather events is the production of CSO. The earliest sewers to prevent flooding were open designs to carry street runoff without treatment away from urban areas and into surface waterways. Open sewers, consisting of gutters and urban streambeds, were common worldwide before the 20th century. In the majority of developed countries, large efforts were made during the late 19th and early 20th centuries to cover the formerly open sewers, converting them to closed systems. However, there was no effort to construct separate sanitary sewage systems because most cities at that time did not have sewage treatment plants and the cost to have two separate sewer systems was cost prohibitive. Therefore, storm water and sanitary sewage were combined into one system called Combined Sewer Systems.

Combined Sewer Systems were typically sized to carry ten to 100 times the average dry weather sewage flows. As sewage treatment plants began to be built, it was infeasible to treat this amount of water, so these plants were typically built to only treat the volume of sewage flowing during dry weather. Diverter structures were installed in the collection system to bypass untreated sewage mixed with surface runoff during wet weather events, protecting sewage treatment plants from overflow, but this practice caused untreated sewage to be released along with storm water during storm events.

Such overflows from Combined Sewer Systems are termed Combined Sewer Overflow (CSO) and when discharged without treatment during wet weather events can cause serious water pollution problems. These discharges contain human and industrial waste, and can cause beach closings, restrictions on shellfish harvesting and consumption, curtailment of recreational use, and contamination of drinking water sources.

CSO is differentiated from Sanitary Sewer Overflow (SSO), which is caused by sewer system obstructions, damage, or flows in excess of sewer capacity often caused by inflow and infiltration of storm water. Absence of a diverter system often causes SSO to flood residential structures and/or flow into natural drainage channels and into the environment untreated. SSO may cause greater health risks and environmental damage than CSO if it occurs during dry weather when there is no rainfall or snow melt to dilute and flush away sewage pollutants.

About 860 communities in the US have Combined Sewer Systems, serving about 40 million people. Pollutants from CSO discharges can include bacteria and other pathogens, oil, toxic chemicals, and debris.

The U.S. Environmental Protection Agency (EPA) issued a policy in 1994 requiring municipalities to make improvements to reduce or eliminate CSO-related pollution problems. In 2000 Congress amended the Clean Water Act to require municipalities to comply with this EPA policy.

Municipalities in the US have been undertaking projects to mitigate CSO since the 1990s. For example, prior to 1990, the quantity of untreated CSO discharged annually to lakes, rivers and streams in southeast Michigan was estimated to be more than 30 billion gallons per year. In 2005, with nearly $1 billion of a planned $2.4 billion CSO investment completed, untreated discharges have been reduced by more than 20 billion gallons per year. This investment in CSO reduction, which included numerous sewer separation, CSO storage and treatment facilities, and wastewater treatment plant improvements have yielded an estimated 85 percent reduction in CSO nationwide.

Many other areas in the US are undertaking similar projects. Cities like Pittsburgh, Seattle, Philadelphia, and New York are under federal consent decrees to solve their CSO issues and face penalties for non-compliance.

Municipalities' sewage departments, engineering and design firms, and environmental organizations offer different potential solutions. Some US cities have undertaken sewer separation projects, which involves building a second piping system for all or part of the community. In many of these projects, cities have been able to separate only portions of their combined systems. High costs or physical limitations may preclude building a completely separate system. In 2011 Washington, D.C. separated its sewers in four small neighborhoods at a cost of $11 million. Another solution is to build a CSO storage facility, such as a tunnel that can store flow from many sewer connections. Because a tunnel can share capacity among several outfalls, it can reduce the total volume of storage that must be provided for a specific number of outfalls. Storage tunnels store combined sewage but do not treat it. When the storm is over, the storm water is pumped out of the tunnel and sent to a WWTP.

Major concerns with the storage of untreated CSO are high capital cost, odors, toxic gases, and removal of settled solids. Some cities have expanded their basic sewage treatment capacity to handle some or all of the CSO volume. In 2002, litigation forced the city of Toledo, Ohio to double its WWTP capacity and build a storage basin in order to eliminate most overflows. The problem with this approach is that the cost to expand the capacity of a WWTP to treat wastewater from storm events is high and often there is not enough space for plant expansion and the construction of storage basins.

In summary, the treatment of wastewater from storm events is a monumental task that has cost billions of dollars in the past with more costs expected in the future. Many municipalities are under consent orders to correct the problem but do not have the financial means to implement standard treatment methods. The most common approach is to contain CSO in basins and tunnels and treat it later at a WWTP but due to the sheer volume of water, this approach often is not possible because of space limitations and limitations in WWTP treatment capacity. This invention describes a cost effective method to utilize the storage capacity of existing water conveyance systems (e.g. piping, ditches, canals and rivers) combined with innovative inline treatment methods to make the treatment of CSO/SSO/Storm Water possible and more affordable by increasing the time for microorganisms to process the pollution contained in the wastewater.

There are approximately 16,000 WWTPs in the US alone with many thousands more worldwide. These WWTPs remove suspended solids and grow beneficial microorganisms in their aeration basins to treat sanitary wastewater.

A WWTP is an ideal factory for growing microorganisms but they are underutilized. They only grow microorganisms that are specifically designed to aerobically treat sanitary wastewater. However, heretofore, they have not been used to grow specialized microorganisms that can pretreat sanitary wastewater in a conveyance system or wastewater resulting from natural catastrophes like hurricanes. Following the process contained in this patent application, WWTPs are enabled to use the ready food source contained in biosolids and convert the biosolids into a food form suitable for growing many different kinds of beneficial microorganisms.

WWTPs are distributed around the country so beneficial microorganisms grown at a WWTP can be used to treat sanitary waste. According to one aspect of the invention, beneficial microorganisms grown at the WWTP can be easily distributed nationwide for other purposes such as providing a biological treatment response to wet weather events like hurricanes or treating sanitary wastes associated with CSO.

The select beneficial microorganisms grown at a WWTP can be also used for disaster relief to kill pathogenic bacteria, reduce pollutants contained in storm water, and kill mosquito larvae growing in stagnant storm water.

Mosquitoes transmit disease and present one of the major health hazards to humans especially in underdeveloped countries. It has been proven that select bacteria can control the growth of mosquito larvae and therefore have a favorable impact on human health.

In summary, to effectively biologically treat pollution contained in wastewater primarily associated with wet weather events, certain conditions are needed all working together synergistically to supply a large supply of select microorganisms grown on an inexpensive supply of food preferably from WWTPs and distributed to the best location that provides ample time for the microorganisms to treat the wastewater. This patent application describes all of these elements that can effectively biologically treat large volumes of CSO and in emergency situations, e.g., in the aftermath of hurricanes, to kill pathogenic bacteria, reduce toxic organics, and kill mosquito larvae.

### OBJECTS AND SUMMARY OF THE INVENTION

Aside from the preferred embodiment or embodiments disclosed below, this invention is capable of other embodiments and of being practiced or being carried out in various ways. Thus, it is to be understood that this invention is not limited in its application to the details of construction and the arrangements of components set forth in the following description and illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

It is therefore an objective of this invention to provide a novel and efficient method to biologically treat wastewater.

Furthermore, it is an objective of this invention to use bioaugmentation (the use of microorganisms to help in the biological treatment of pollution), which includes bacteria and phages (viruses) in the treatment of wastewater.

Furthermore, it is an objective of this invention that bioaugmentation includes a mixture of ideally selected microorganisms to biologically treat wastewater, preferably a group of facultative bacillus bacteria that are film-forming.

Furthermore, it is an objective of this invention to use bioaugmentation to treat wastewater in its conveyance system, which may include pipelines, canals, and streambeds, in order to provide sufficient residence time for effective biological treatment.

Furthermore, it is an objective of this invention to add bioaugmentation to the conveyance system as close to the source of pollution as possible but preferably at the source.

Furthermore, it is an objective of this invention to grow the mixture of select microorganisms near to a source of organic material as food for the microorganisms and near to the source of pollution.

Furthermore, it is an objective of this invention to use biosolids produced at a WWTP to grow select microorganisms for use within the WWTP and to deliver the microorganisms into the wastewater conveyance system via a pipeline contained within the conveyance system.

Furthermore, it is an objective of this invention to disinfect the biosolids from a WWTP and convert them into a food source to grow selected beneficial microorganisms used to biologically treat wastewater.

Furthermore, it is an objective of this invention to add one or more flow control devices to increase the holding capacity of the wastewater conveyance system, which will allow more time for the mixture of select microorganisms to perform their water treatment functions.

Furthermore, it is an objective of this invention for the flow control device to also level out the flow rate of CSO and storm water from the conveyance system and other retention facilities so this water can be slowly clarified and disinfected if necessary over an extended period of time.

Furthermore, it is an objective of this invention to affix biocarriers to the inner surfaces of the wastewater conveyance system or to place the biocarriers anywhere within the conveyance system to enhance the growth of beneficial biofilm.

Furthermore, it is an objective of this invention to provide a screening system to remove large and floatable solids from wastewater prior to clarification and disinfection.

Furthermore, it is an objective of this invention to use high-rate clarification and flocculating polymers, preferably in a system that uses magnetite as a ballast material in a high-rate clarification system, to remove fine suspended solids contained in wastewater so that treated wastewater can be directly discharged into the environment without the need of a WWTP.

Furthermore, it is an objective of this invention for the wastewater first entering the conveyance system at the beginning of a storm event, which typically is the most contaminated and accordingly termed “first flush”, to be diverted into the WWTP for immediate treatment.

Furthermore, it is an objective of this invention to irrigate land with water and suspended solids coming from the high-rate clarification system.

Furthermore, it is an objective of this invention to provide additional storage and treatment capacity in structures located in a stream or riverbed to supplement the treatment capabilities of the existing conveyance system.

Furthermore, it is an objective of this invention to use biosolids, especially the microorganisms collected from the high-rate clarification system, to enhance biological treatment capacity of a WWTP.

Furthermore, it is an object of this invention to use WWTPs as a means of producing select microorganisms to be used to treat bacteria contaminated water and mosquito larvae resulting from hurricanes or other extreme storm events.

Certain distinct elements of the system of this invention are known individually in the art, such as the use of inline bioaugmentation and high-rate clarification. However, these known elements have never been combined with other elements provided according to the invention, such as increasing the holding capacity of the conveyance system by the use of flow control devices or growing select microorganisms at a WWTP and delivering the select microorganisms to the head of the conveyance system, to produce a unique combination of treatment elements to effectively biologically treat wastewater. By proper combination of these elements, this invention provides a system that can cost-effectively biologically treat wastewater.

More specifically, Dickerson in U.S. Pat. No. 5,788,814 discloses a bioaugmentation method for treating wastewater in its conveyance system prior to a WWTP. The Dickerson patent was the basis for starting a company named In-Pipe Technology, Inc. In-Pipe Technology uses the continuous addition of an extremely high concentration of microbes into the collection system to eliminate sewer system grease and odor problems, reduce sludge production, reduce electrical power requirements at the WWTP and to reduce infrastructure corrosion.

Dickerson did not contemplate growing the bacteria at a WWTP using biosolids produced at the WWTP, nor contemplate the delivery of bacteria from the WWTP via a pipeline contained inside the conveyance system, nor contemplate provision of flow control devices to hold up the flow of wastewater in the conveyance system to increase the residence time for biological treatment.

Williams et. al. U.S. Pat. No. 8,828,229 discloses a method of treating wastewater using a membrane biological reactor (MBR) to reduce the hydraulic load on a WWTP by providing a concentrated organic waste stream to the WWTP. The Williams patent describes the function of the MBR as an intermediate WWTP that biologically treats the wastewater in the conveyance system upstream from the WWTP so that clean water can be discharged and the concentrated biosolids flow to the WWTP for additional treatment. The Williams patent describes the benefit of this process for the treatment of municipal sanitary wastewater and does not apply to the treatment of inline biological treatment of wastewater. As noted above, the quantity of municipal sanitary wastewater increases during rain events, during which the treatment method expressed in the Williams patent would reduce the hydraulic load on the WWTP, but this is only accomplished by the use of membrane bioreactors positioned along the conveyance system. Williams' patent neither contemplates increasing the holding capacity of the conveyance system nor does it use high-rate clarification followed by disinfection if necessary for direct discharge of treated wastewater without the need for a WWTP.

Tests by SCD Probiotics in Cartagena, Colombia, South America proved that the use of probiotics in a conveyance system increased the efficiency of sewage treatment by extending the treatment time. In a sewage system 21 Km in length and having a typical residence time of 2.5 hours, TSS was reduced 68% and BOD reduced 66%

Another test by SCD Probiotics in Lowicz, Poland using probiotics showed a reduction of BOD from 623 ppm to 13.2 ppm with an overall 25% reduction in sludge production when sewage was treated inline.

The preferred bacteria contained in the mixture of microorganisms used in this patent are in the family of facultative bacillus that are non-pathogenic and non-toxic, which includes but is not limited to B. Subtiles, B. Licheniformes, B. Indicus, B. Coagulans, B. Cereus, and B. Clausii. These bacteria are ubiquitous in nature, especially in soil. They are facultative by nature, meaning that they do not need oxygen to survive and to be productive. Therefore they are effective in both aerobic and anaerobic environments and reduce odor and corrosion in the conveyance systems by consuming chemicals contained in the wastewater. If the mixture of facultative microorganisms flows to a WWTP, the need for aeration is reduced or completely eliminated and electrical usage can be reduced by as much as 50%, because most WWTPs use aerobic bacteria that need high levels of oxygen and therefore incur high electrical costs for aeration.

Phages are viruses that infect and kill microorganisms. Sulfur reducing bacteria (SRB) cause the most harm in a water conveyance system because they convert sulfates contained in the water into sulfides that react to form hydrogen sulfide, a major cause of odor, and sulfuric acid, a major cause of corrosion. Phages infect and control the growth of SRB but can be selected to do so while not harming beneficial microorganisms.

The bioaugmentation performed in the conveyance system requires microorganisms that are either grown onsite or offsite. The preferred embodiment of this invention is to produce microorganisms at a WWTP, using biosolids in waste water as food for the microorganisms.

A second aspect of the treatment process according to the invention is control of the flow of the wastewater to be treated to increase the holding capacity of the wastewater conveyance system, which will enhance the performance of inline bioaugmentation as discussed above. That is, during both dry and wet weather conditions, the flow control device is operated so as to control the residence time of wastewater in the conveyance system to provide sufficient time for the microorganisms to perform their function.

Flow control can take many forms but the preferred method is to provide a damming device containing an adjustable weir to control the residence time of the select microorganisms in the conveyance system, which can be an existing device converted according to the invention into a treatment system. This gives the microorganisms adequate time to act to reduce nutrient levels. The importance of flow control in a conveyance system results from the fact that the flow during wet weather events can be 10 to 100 times the flow in the conveyance system during dry weather. Without delaying some of the storm flow using a variable-height weir, the storm flow will be too great to allow the microorganisms enough time to biologically treat the wastewater. Another important function of the flow control device is to level out the flow of wastewater over a period of time so that the wastewater can be clarified and disinfected at a slower rate over a longer period of time. It is also within the scope of the invention, in order to increase the residence time for biological treatment and to save space, to extend the wastewater conveyance system along the edge of a riverbed like a canal. In some cases the wastewater conveyance system can actually be positioned in the riverbed.

The third aspect of treatment according to the invention is high-rate clarification followed by disinfection if necessary. Clarification is necessary to meet total suspended solids (TSS) limits and since the TSS levels influence basic oxygen demand (BOD), which is a measure of the amount of organics present, and bacteria levels, fine suspended solids must be removed. The most effective method for fine solids removal is high-rate clarification using flocculating polymers. Because of the high flow rates during storm events, only high-rate clarification is applicable, primarily because of space and cost limitations. This invention contemplates the use of high-rate clarification methods that have a surface overflow rate (SOR) greater than 10 gallons per minute per square foot of surface area of the clarifier. For example, for a flow rate of 10 million gallons per day or about 7000 gallons per minute, the surface area of a high-rate clarifier with a SOR of 10 gallons per minute per square foot would need to be about 700 square feet. By comparison, if a traditional gravity clarifier with a SOR of one gallon per minute per square foot were used the clarifier would need to have a surface area of about 7000 square feet. Therefore, a significant saving in the space required is realized. The methods of high-rate clarification methods preferred for practice of this invention are dissolved air flotation, lamella gravity clarification, and ballast clarification using sand or magnetite, all of which are generally well understood in the art.

High-rate clarification produces a concentrated slurry of suspended solids in the approximate range of 1-5 weight % solids. This slurry can be dewatered by mechanical means to produce a dry cake that can be either landfilled or land applied. Another economical solution is to flow the concentrated slurry into a dewatering containment that will allow adequate time for the solids to settle and then be removed periodically as a more concentrated wet solid. This wet solid can be land applied but not landfilled unless it passes a paint filter drip test. Supernatant from the dewatering containment can be directly discharged, assuming the total suspended solids are low enough to meet regulatory limits. However the best solutions are to apply the concentrated slurry from the high-rate clarification system or the dewatering containment directly to the land to improve soil conditions, or to a WWTP to increase biological treatment capacity, assuming that regulatory limits are not exceeded.

Because of the sheer magnitude of the problem and the cost of adequate solutions, regulations regarding wastewater are constantly changing. It appears, according to present EPA regulations, that primary clarification and disinfection are acceptable solutions to the problem if bacteria levels can be sufficiently low and the BOD of the wastewater can be reduced by 85%. This may not be possible unless bioaugmentation in the conveyance system and containment structures is followed by flow control to increase the capacity of the conveyance system to achieve the necessary level of biological treatment, in turn followed by high-rate clarification using flocculating polymers according to this invention.

The combination of the treatment methods contained in this invention is arranged in such a way to meet regulation-specified discharge limits, which primarily specify acceptable levels of TSS, BOD, bacteria, and floatables. The level of BOD and bacteria are reduced significantly by the inline bioaugmentation within the conveyance system but if the bacterial levels are still too high, further disinfection may be performed as necessary, using methods including but not limited to employment of known disinfectants such as chlorine, peracetic acid, UV, and ozone.

WWTPs grow bacteria that are beneficial in the treatment of municipal wastewater. These bacteria use the organic wastes and other chemicals contained in municipal wastewater (CSO) as a food source, thus consuming the wastes and purifying the water. Since this food source is basically free and WWTPs are located around the country, a WWTP makes an ideal factory for growing beneficial bacteria for inline treatment of CSO, improvements to their own treatment process, or for the digestion of biosolids produced in the wastewater treatment process according to the invention.

The beneficial microorganisms grown at a WWTP can also be used for other environmental purposes that include but are not limited to disaster response to storm events such as hurricanes.

In addition to the adverse effects on WWTP operations, storm events cause major problems with microorganism contamination, mold production, toxic organic contamination, and mosquitoes. Beneficial microorganisms grown at a WWTP can be used to address all of these problems. For example, the efficiency of Bacillus Thuringiensis Israelensis (BTI) and Bacillus Sphaericus have been reported in a paper by Lacey L A, J. Am. Mosquito Control Association, 2007; 23 (2 Suppl); 133-63 to be effective in a variety of habitats against multiple species of mosquitoes. Also, the EPA documents that BTI is used across the United States for mosquito control and is approved for aerial spraying. BTI has been shown to be effective in reducing mosquito larval population and could be effective in controlling mosquitoes carrying Zika, Dengue, and Chikungumya viruses. The most effective way to use BTI to control mosquitoes is to grow BTI at a WWTP in large quantities to be applied globally.

Beneficial microorganisms are best grown in conditions with a readily available food supply that does not contain competing microorganisms like pathogenic bacteria found in municipal solid wastes. Therefore, it is preferred to disinfect the solid waste supplied to the WWTP to convert the solid waste into a clean food supply, so that desirable microorganisms can grow without competition from undesirable microorganisms.

While there may be other methods for disinfecting and making a food supply ideal for growing microorganisms, the preferred embodiment of the present invention is to use hydrodynamic cavitation for these purposes. Hydrodynamic cavitation involves agitating water with high energy to form bubbles. When the bubbles subsequently collapse, extremely high temperature and pressure conditions are created, which destroy undesirable microorganisms which would otherwise compete with the select beneficial microorganisms, which are introduced after the hydrodynamic cavitation step. Hydrodynamic cavitation also will lyse cells to destroy the cell wall and release liquid protoplasm, which is an ideal liquid food for growing microorganisms.

The effectiveness of a biological treatment system is a function of the type of microorganisms and the conditions that foster the growth of a healthy population of microorganisms. The two basis types of microorganisms are mobile organisms that are free floating and fixed microorganisms that become attached to some solid surface. The effectiveness of a biological treatment system is improved when there is a combination of both mobile and fixed microorganisms. Increasing the surface area for fixed microorganisms to attach to increases the concentration of microorganisms and therefore the effectiveness of the biological treatment process. Surface area is increased by the addition of biocarriers that are usually made of plastic with a high surface area and can be either fixed or floating. To increase the ability of a conveyance system to act as a biological reactor requires the attachment of fixed biocarriers to the inside of the conveyance system so the microorganisms can remain in the conveyance system to accomplish biological treatment.

### BRIEF DESCRIPTION OF THE DRAWINGS

Other objects, features and advantages will occur to those skilled in the art from the following description of a preferred embodiment and the accompanying drawings, in which:

FIG. 1 shows an overview of the treatment technologies employed according to the invention and how they are integrated to produce the desired result of economically treating wastewater.

FIG. 2, comprising FIGS. 2(a) and 2(b), shows a flow control device that can be adjusted to change the pool level in the conveyance system, which will vary the holding capacity of the conveyance system.

FIG. 3 shows a system for growing a mixture of preferred microorganisms at a WWTP to bioaugment the inline treatment of wastewater.

FIG. 4 shows the extension of a wastewater conveyance system and its placement inside a riverbed.

FIG. 5 shows a delivery system that takes select microorganisms grown at a WWTP and transports the select microorganisms either through a pipeline placed within the wastewater conveyance system or for other environmental purposes.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles of the invention, and are not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

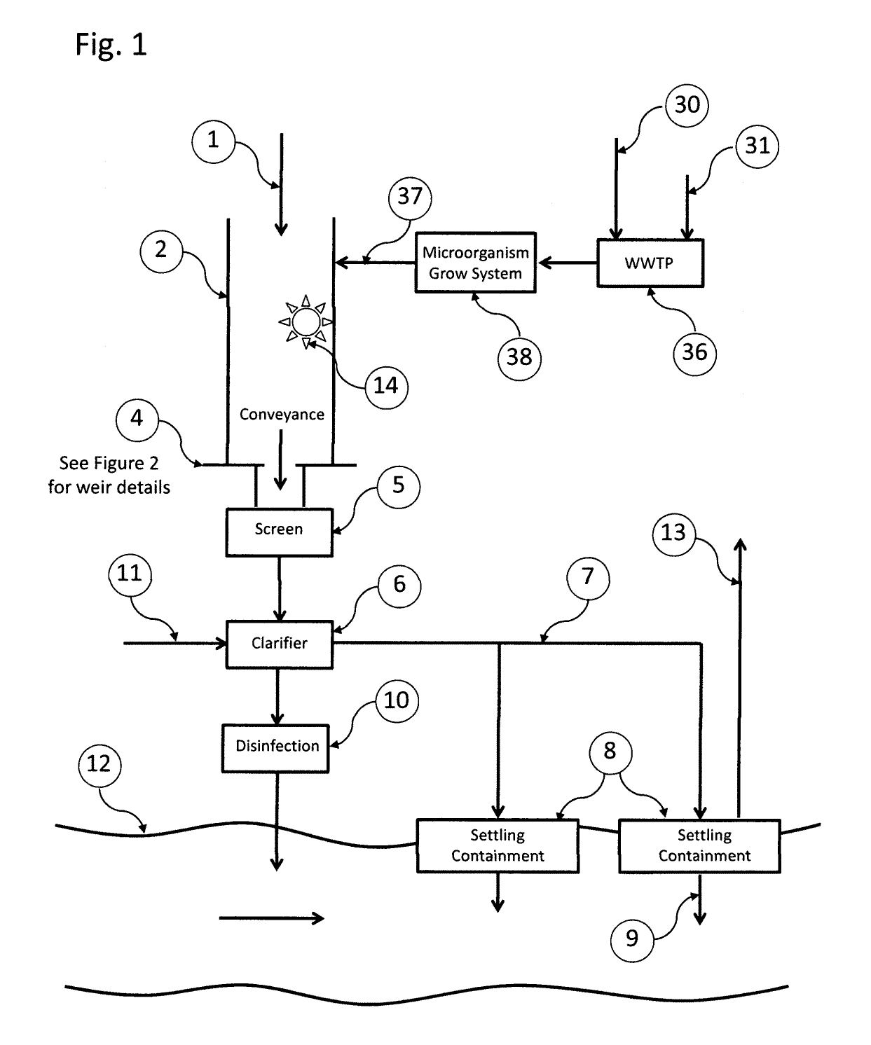

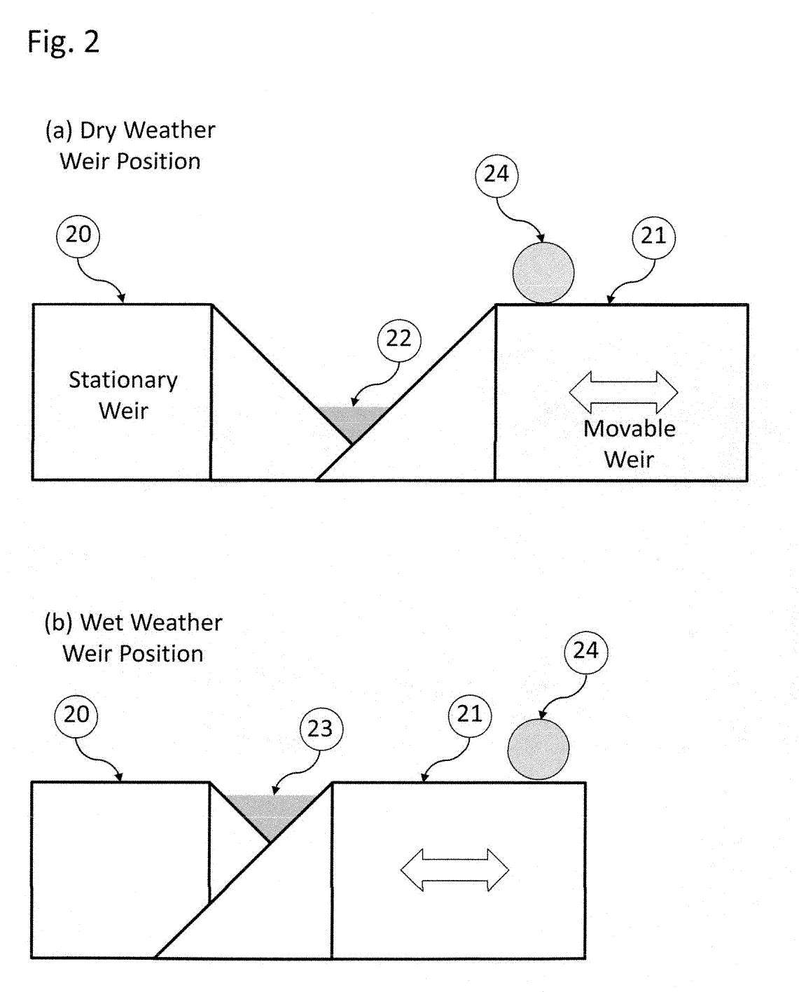

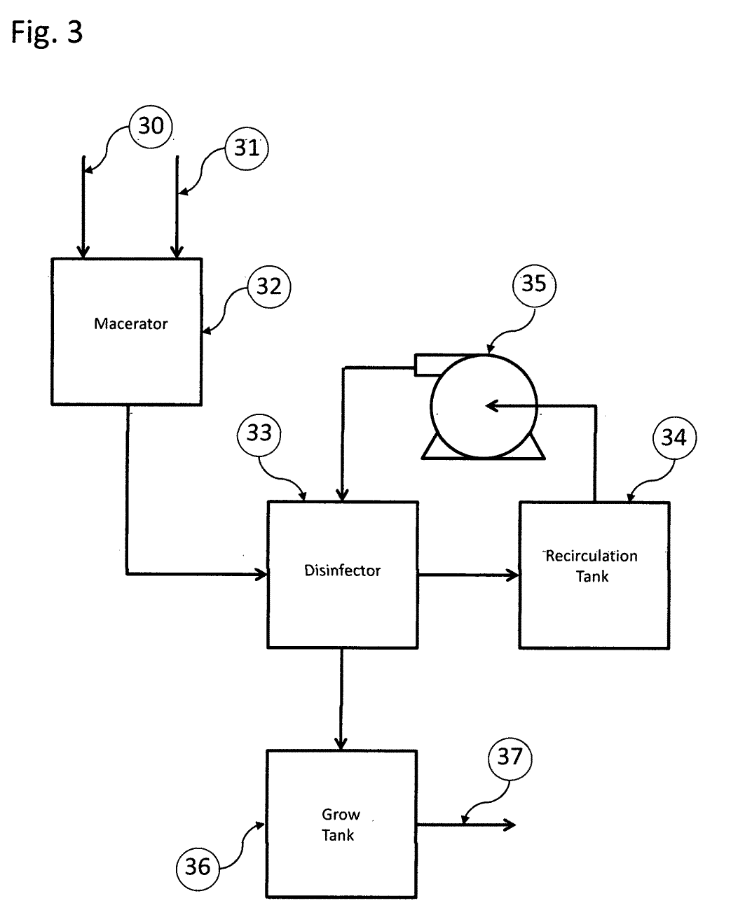

FIG. 1 shows wastewater (1) flowing through a water conveyance system (2), such as a lake, river, impoundment structure or the like, into which is injected a mixture of select microorganisms (37) that treat the wastewater inside the water conveyance system (2) to consume undesirable organics. As indicated above, these microorganisms may include facultative bacilli and phages. The water conveyance system (2) that contains the flowing stream of wastewater (1) contains a plethora of biocarriers (14) that are attached to the inside surface of the conveyance system (2) to increase the amount of surface area for the growth of biofilm. Also injected at or near the beginning of the conveyance system (2) is a mixture of select microorganisms (37) that are grown in a growth system (38) shown in more detail in FIG. 3 that uses organic wastes (30) from a WWTP (36) and other organic wastes (31). A flow control device (4), shown in more detail in FIG. 2 is located at the end of the conveyance system (2) to control the flow rate and the level of wastewater in the water conveyance system (2) to give the microorganisms time to work. Biologically treated wastewater then flows through a screen device (5) that removes large solids and floatables. The screened wastewater then flows into a high-rate clarification device (6), which may take one of several forms as mentioned above, but preferably where polymer (11) is added to flocculate fine suspended solids for removal. Flocculants may be employed together with magnetite to remove the flocculated solids, e.g., as described in applicant's co-pending application Ser. No. 14/612,635, filed Feb. 3, 2015, and incorporated herein by this reference. The clarified wastewater then flows through a disinfection device (10) and then into the end conveyance, e.g., a river (12). Solids removed by the high-rate clarification device flow (7) into one or more solids settling containments (8). Supernatant from the solids settling containment (8) flows (9) into the river (12) or is land applied. Solids dredged from the solids settling containment (8) from time to time are either land applied or land filled (13).

FIG. 2, comprising FIGS. 2 (a) and (b), shows a flow control device (4) that can be adjusted to change the pool level in the conveyance system. The main purpose of the flow control device is to back up wastewater into the conveyance system to allow for additional time for biological treatment and to control and level out the flow of wastewater for subsequent treatment. The preferred flow control device is a V notch weir composed of two parts. One part (20) is a stationary weir and the other part is a movable weir (21) sealed to the stationary weir to prevent most leakage. The flow control device has basically two positions. One position (FIG. 2(a)) determines a low-level pool height (22) that is employed during dry weather and another position (FIG. 2(b)) determines a high-level pool height (23) that is employed during wet weather events. This allows the residence time of water in the conveyance system to be controlled to be substantially constant despite varying flow rates in dry and stormy weather. A mechanical device (24) powers the movable weir (21).

FIG. 3 shows a system for growing a mixture of select microorganisms comprised primarily of bacteria and phages to bioaugment the inline treatment of wastewater. Organic waste (30), specifically from a WWTP and other organic wastes (31) are macerated in a device (32) designed to blend and size reduce solids contained in the organic wastes (30 and 31). This blended product flows into a disinfector (33) that disinfects the organic wastes so that microorganisms contained in the organic wastes do not compete with the growth of the select mixture of microorganisms used in the treatment of wastewater. The preferred method to disinfect the organic wastes is by hydrodynamic cavitation, which also lyses cells contained in the organic wastes to improve the food value for the growth of the select microorganisms. The organic wastes that have been treated with hydrodynamic cavitation then flow into a recirculation tank (34) and are pumped (35) back to the disinfector to allow the organic wastes to be disinfected and lysed multiple times. Once suitably treated, the organic wastes flow to a grow tank (36) to feed the select mixture of microorganisms, preferably facultative bacillus bacteria and phages as discussed above. These microorganisms then flow (37) into the wastewater conveyance system (2) shown in FIG. 1.

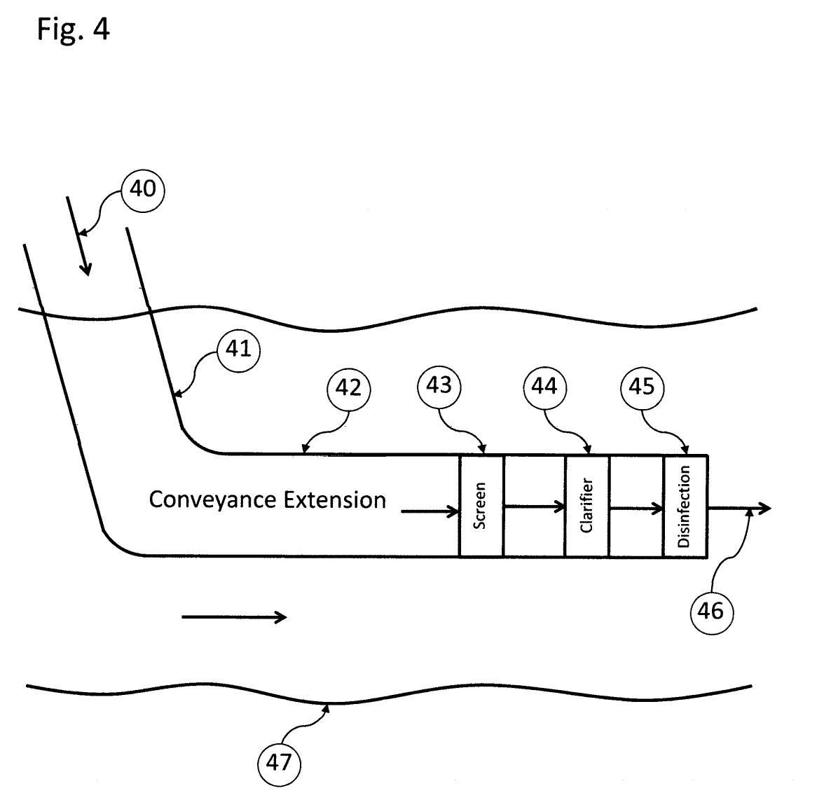

FIG. 4 shows wastewater (40) flowing through a conveyance system (41) and into a conveyance system extension (42) that has been positioned within a riverbed and where biological treatment is taking place. The biologically treated wastewater within the conveyance system extension (42) is then screened (43) to remove floatables, then clarified (44) to remove suspended solids, and then disinfected (45) to kill pathogens before the treated water is discharged (46) into a riverbed (47). First the water is biologically treated in the conveyance system and then it is screened, then clarified, then disinfected before it is discharged into the river water.

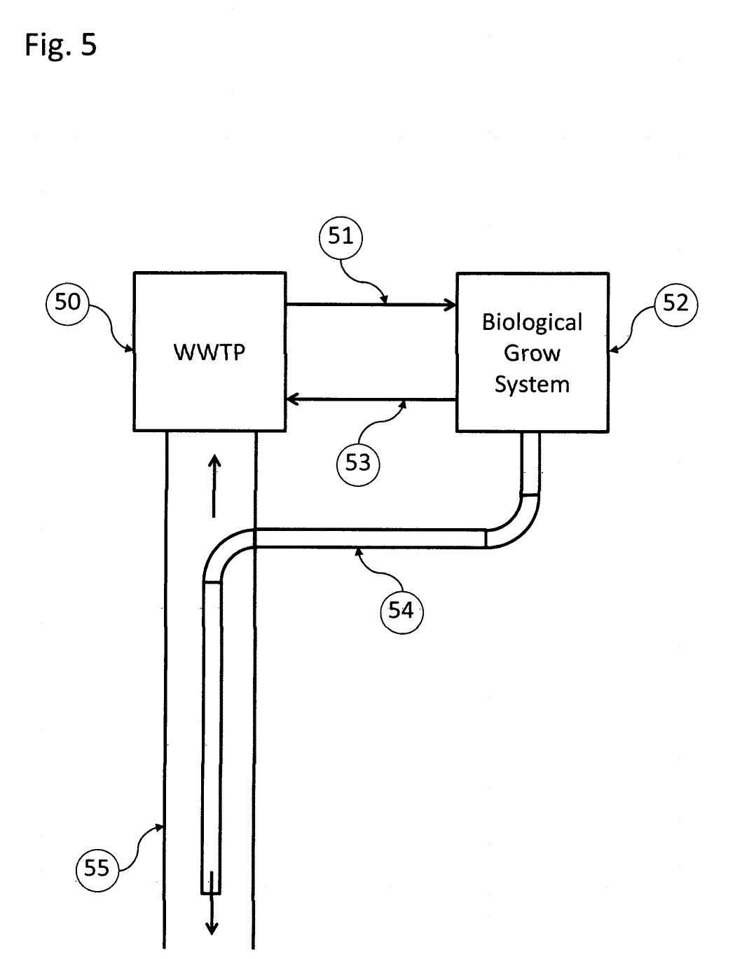

FIG. 5 shows a biological grow system (52) that receives organic wastes (51) from a WWTP to produce select microorganisms that are either returned (53) to the WWTP (50) for use and piped (54) to the front end of the wastewater conveyance system (55) for inline bioaugmentation or used (56) for other environmental purposes.

While a preferred embodiment of the invention has been disclosed, the invention is not to be limited thereto, but only by the following claims.

What I claim as my invention is:

1. A method for treating a stream of polluted wastewater by:
growing select microorganisms at a Waste Water Treatment Plant (WWTP) using properly treated solid and liquid wastes generated or received at the WWTP along with other wastes;
introducing a mixture of the select microorganisms into an inlet end of a wastewater conveyance system to convert said conveyance system into a treatment system able to inline destroy detrimental microorganisms, treat toxic organics, reduce BOD, and to remove nutrients from the stream of wastewater;
providing an adjustable flow control device in said wastewater conveyance system to allow control of the accumulation of wastewater within the conveyance system, so as to control the residence time of the stream of wastewater in the conveyance system; and
providing a high-rate clarification treatment that processes wastewater that has been biologically treated in the conveyance system to meet discharge limits and final use requirements.

2. The method of claim 1, wherein the mixture of select microorganisms contains free floating and film forming bacteria and phages that have an adverse effect on detrimental bacteria such as SBR bacteria and other microorganisms.

3. The method of claim 1, wherein the mixture of select microorganisms includes Bacillus Thuringiensis lsraelensis and Bacillus Sphaericus that are used to kill mosquito larvae.

4. The method of claim 1, wherein the preferred select mixture of microorganisms contains bacillus bacteria selected from the group including B. Subtiles, B. Licheniformes, B. Indicus, B. Coagulans, B. Cereus, and B. Clausii.

5. The method of claim 1, wherein the mixture of select microorganisms is grown at or near the site of entering pollution into the conveyance system using nutrients found in the pollution or other organic wastes as a food source.

6. The method of claim 1, wherein attached biocarrier surfaces are added to the conveyance system, which includes pipelines, canals, river, and streambeds, to promote the growth of select microorganisms as biofilms.

7. The method of claim 1, wherein the conveyance system may be a pipeline, canal, or a natural watercourse that is extended by a man-made canal or other conveyance system added alongside or within a river or stream to increase the residence time so biological treatment can continue to treat wastewater before entering the river or stream.

8. The method of claim 1, wherein biosolids or other wastes received from a WWTP are disinfected and lysed to produce a suitable food to grow the select microorganisms.

9. The method of claim 8, wherein the waste products to grow the select microorganisms are disinfected and lysed by methods including but not limited to mechanical, thermal, electrical, sonic methods, but preferably hydrodynamic cavitation, to kill unwanted competing microorganisms, including pathogenic bacteria and sulfur reducing bacteria (SRB) and to increase the food value of the waste products by releasing the cell protoplasm contents beneficial for growing the select microorganisms.

10. The method of claim 1, wherein the flow control device comprises an adjustable weir so as to create either a smaller reservoir of wastewater during dry weather conditions or a larger reservoir of wastewater during wet weather conditions, allowing control of the residence time of the wastewater in the reservoir to provide optimum time for the select microorganisms to treat the wastewater to appropriate levels.

11. The method of claim 10, wherein the flow control device controls and levels out the flow of wastewater from the conveyance system so that the flow does not exceed the capacity of any downstream treatment system.

12. The method of claim 10, wherein the flow control device is a “V” notch weir that can be mechanically adjusted to control the rate of flow of wastewater discharged from the conveyance system.

13. The method of claim 1, comprising the further step of screening floatables and disinfection after said high rate clarification step.

14. The method of claim 1, wherein the high-rate clarification system removes fine suspended solids including bacteria and other microorganisms by the use of a flocculating polymer and/or coagulant followed by disinfection when needed.

15. The method of claim 14, wherein the high-rate clarification system has a surface overflow rate greater than 10 gallons per minute per square foot and employs techniques selected from a group of technologies including dissolved air flotation, lamella gravity clarification, densified sludge, and ballast clarification using either sand or preferably magnetite.

16. The method of claim 14, wherein solids removed by the high-rate clarifier and any first flush wastewater are diverted into one or more containment structures that are either positioned in a riverbed that increases the biological treatment time or pumped to a nearby WWTP for further biological or dewatering treatment.

17. The method of claim 13, wherein when necessary, disinfection methods will be employed including treatment with chlorine, peracetic acid, UV, or ozone to kill pathogenic microorganisms in biologically treated wastewater before direct discharge into the environment.

18. The method of claim 14, wherein select microorganisms that have been removed by the high-rate clarifier are routed to a WWTP to provide a constant source of the select microorganisms to enhance the performance of the WWTP and to treat the wastewater in the conveyance system downstream of the WWTP when needed.

19. The method of claim 1, further comprising the steps of using the select beneficial microorganisms grown from municipal wastewater; for enhancing the performance of a WWTP; improving digesting of WWTP biosolids; treating CSO/SSO/Storm Water inline; treating toxic organics contained in wastewater from a wet weather event; and killing mold.

20. The method of claim 1, wherein the select microorganisms grown at the WWTP are pumped through a new pipeline positioned inside the conveyance system to the front end of the conveyance system to increase the residence time for biological treatment.
